(19) United States
(12) Patent Application Publication
(10) Pub. No.: US 2006/0178341 A1
(43) Pub. Date: Aug. 10, 2006

(54) Composition comprising soluble glucan oligomer from saccharomyces cerevisiae is2 inhibiting the swine influenza (SIV) and transmissible gastroenteritis coronavirus (tgev)

(76) Inventors: Won Kook Moon, Seoul (KR); Dong Woo Kim, Seoul (KR); Jeong Hoon Park, Daejeon (KR); Bong Hyun Chung, Daejeon (KR)

Correspondence Address: KIRK HAHN, 14431 HOLT AVE, SANTA ANA, CA, 92705, US

(21) Appl. No.: 10/561,714

(22) PCT Filed: Jun. 23, 2004
(86) PCT No.: PCT/KR04/01515

§ 371 Date: Dec. 21, 2005

(30) Foreign Application Priority Data
Jun. 23, 2003 (KR) 10-2003-0040535

(51) Int. Cl. A61K 31/716; A61K 36/06

(52) U.S. Cl. 514/54; 424/195.16

(57) ABSTRACT

The present invention relates to the soluble glucan oligomer having a M.W. ranging from 1,000 to 10,000 prepared by treating insoluble beta-glucan isolated the cell wall of yeast variant IS2 with commercially available beta-glucan hydrolyzing enzymes. The soluble glucan oligomer showed potent efficacy on inhibiting activity of influenza virus and transmissible gastroenteritis coronavirus, therefore, it can be used as the therapeutics or health care food for treating and preventing mammal's diseases caused by the infection of influenza virus and transmissible gastroenteritis coronavirus.

### TECHNICAL FIELD

The present invention relates to the composition comprising soluble glucan oligomer isolated from Saccharomyces cerevisiae IS2 inhibiting the swine influenza (SIV) and transmissible gastroenteritis coronavirus (TGEV).

### BACKGROUND ART

Influenza has been a major cause of morbidity in human and of mortality in the elderly and infant. Influenza viruses are members of the family Orthomyxoviridae, which is composed of four genera, i.e., influenza virus A, B, C and Thogotovirus. Clinical features of influenza include high fever, chill, cough, sore throat, runny or stuffy nose, headache, myalgia and often extreme fatigue. Most of patients infected with influenza virus recover completely within one or two weeks. However, a number of people are suffered with further developed serious complications and died from the complications. There have been several recurring pandemics till now all over the wold, for example, Asian flu (H2N2) in 1957, Hong Kong flu (H3N2) in 1968, Russian flu (H1N1) in 1977 and recently overwhelming Hong Kong SARS (Severe Acute Respiratory Syndrome) influenza outbreak in 2003, which drives all the Asian people to fear of death (de Jong J C., et al., Nature, 389, pp 554, 1997; Subbarao K., et al., Science, 279, pp 393-396, 1998; Claas E C., et al., Lancet, 351, pp 472-477, 1998; Yuen K Y., et al., Lancet, 351, pp 467-471, 1998). There have been known that influenza is originated from four types of influenza mixovirus, i.e., A, B, C₄ type virus and corona virus. Although all the viruses shows similar clinical feature, one vaccine against a virus doses not have immunity to the other types of influenza viruses.

As a one of prevention methods, the viral vaccine of influenza has been recommended and it has been known to show 70-80% preventing activity (Influenza, Plenum Medical Book Company, p 291, 1987). However, since the vaccine endows short duration of immunity and is provided with injection, it has several problems such as difficulty in administration into children and in initial prevention of influenza.

Diarrhea of pigs, especially in sucking and weaned piglets caused by infectious virus or microorganism results in large economic losses in pig breeding farms. Enteric viruses possess unique characteristics in respect to their intestinal tropism and replication (Saif L. J., et al., Disease of swine 8^{th}, Iowa State University Press, Ames., USA, 1990). Most of the enteric viruses have heat labile property which gives rise to the prevalence of viral diarrheas during the winter.

Corona virus, an aetiological virus of SARS is known to be transferred from animal to human as a mutant type and is a principle virus to cause to give rise to the diarrhea of pigs, to lose the appetite of pig, which result in inhibiting the growth of pig. Furthermore, it could not treated by conventional antibiotics and basic treating therapy has not been developed yet till now. Transmissible gastroenteritis (TGE), a member of the coronaviridae is an economically important disease because it is highly contagious and characterized by vomiting, severe diarrhea and high mortality in piglets during the first few weeks of life. It has been reported that the virus had been found all over the world including Korea.

Beta-glucan can be isolated from various resources such as yeast, microorganism, mushroom, grain and algae. It has been studied and applied as various types of product till now. In particular, beta-glucan derived from yeast cell wall has been studied and known well.

Yeast, a microorganism classified into GRAS (Generally Recognized As Safe) in FDA, has been used in various field including food field and the inner cell membrane of yeast comprises beta 1,3- and 1,6-glucan as main ingredients, and a small amount of chitin and mannoprotein, however, outer cell membrane thereof comprises mannoprotein, a protein linked to mannan.

Beta-glucan, a major component of yeast cell wall, has been reported to increase Ag-specific immune response by activation and proliferation of macrophage, to elevate the resistance to pathogen such as fungi, bacteria, virus and the like, to inhibit the immune depression observed in trauma and to increase resistance to cancer or cancer metastasis in a host (Abel, G. and Czop, J. K., Int. J. Immunophamacol., 14, pp 1363-1373, 1992; Babineau, et al., 220(5), pp 601-609, 1994; Benach J. L., et al., Infection and Immunity, 35(3), pp 947-951, 1982; Di Renzo, L., et al., Eur. J. Immunol., 21, pp 1755-1758, 1991; Fukase, S., et al., Cancer Res., 47, pp 4842-4847, 1987; Janusz, M. J., et al., J. Immun., 142, pp 959-965, 1989; Olsen, E, J., et al., J. Immun., 64, pp 3548-3554, 1996, Sakurai, T., et al., Int. J. Immunopharmacol., 14, pp 821-830, 1992; Czop, J. K., et al., Prog. Clin. Biol. Res., 297, pp 287-296, 1989).

Since beta-glucan of yeast is a water-insoluble polysaccharide, a number of preparation methods to obtain beta-glucan with high solubility have been developed till now as follows. U.S. Pat. No. 5,576,015 discloses the method of preparing beta-glucan with a form of fine particle to increase its absorption rate; U.S. Pat. No. 4,877,777 discloses the method of introducing chemical formula into glucan to increase its solubility; U.S. Pat. No. 5,037,972 and U.S. Pat. No. 6,143,883 disclose the method of preparing soluble glucan particles by extracting glucan with organic solvent and subsequently treating with beta-glucanase or cellulase which can degrade beta-1,3-D-glucose chain, a basic structure of the glucan.

However, there has been not reported or disclosed about the specific soluble glucan oligomer isolated from yeast variant strain IS2 (KCTC 0959BP) and the therapeutic effect for influenza virus and transmissible gastroenteritis coronavirus disease of the glucan oligimer in any of above cited literatures, the disclosures of which are incorporated herein by reference. The inventors of the present invention have been endeavored to find pharmacologically potent beta-glucan from specific yeast variant strain from investigate and finally completed present invention by confirming that the soluble glucan oligomer having less than 50,000 D of M. W. obtained by extracting the cell wall of yeast mutant IS2 shows potent inhibiting activity of influenza virus and transmissible gastroenteritis coronavirus.

These and other objects of the present invention will become apparent from the detailed disclosure of the present invention provided hereinafter.

### SUMMARY OF THE INVENTION

According to one aspect of the present invention, the present invention provides the pharmaceutical composition comprising soluble glucan oligomer derived from yeast variant strain IS2 (KCTC 0959BP) for treating and preventing the diseases caused by the infection of influenza virus and transmissible gastroenteritis coronavirus in human and mammal.

According to another aspect of the present invention, the present invention provides a use of a soluble glucan oligomer derived from yeast variant strain IS2 (KCTC 0959BP) for the preparation of therapeutic agent for treatment and prevention of the diseases caused by the infection of influenza virus and transmissible gastroenteritis coronavirus in human and mammal.

### DISCLOSURE OF THE INVENTION

Accordingly, it is an object of the present invention to provide the pharmaceutical composition comprising soluble glucan oligomer derived from the cell wall of yeast variant strain (KTCT 0959BP) obtained by treating insoluble beta-glucan with enzyme, for treating and preventing the diseases caused by the infection of influenza virus and transmissible gastroenteritis coronavirus in human and mammal.

It is an object of the present invention to provide a use of a soluble glucan oligomer derived from yeast variant strain IS2 (KCTC 0959BP) for the preparation of therapeutic agent for treatment and prevention of the diseases caused by the infection of influenza virus and transmissible gastroenteritis coronavirus in human and mammal.

Above described influenza virus comprises influenza virus A, B, C₄ and swine influenza virus.

Above described disease caused by the infection of influenza virus comprises influenza, common cold, laryngopharyngitis, bronchitis, pneumonia, and the like.

Above described disease caused by the infection of transmissible gastroenteritis coronavirus comprises common cold, severe acute respiratory disease, porcine epidemic diarrhea (PED), transmissible gastro enteritis(TGE), and the like.

Also, above described mammal includes domestic animals such as dog, cat, cattle, pig and so on.

It is another object of the present invention to provide the method for preparing soluble glucan oligomer comprising the steps consisting of: (a) culturing yeast (Saccharomyces cerevisiae) variant IS2 (KCTC 0959BP) in the culture broth for inoculation; (b) inoculating above yeast culture solution to culture broth, culturing and centrifuging to obtain yeast; (c) adding NAOH thereto to extract beta-glucan from yeast cell wall; (d) reacting extracted beta-glucan with hydrolyzing enzyme and then subjecting to filtration to obtain soluble glucan oligomer; and (e) finally drying with lyophilization to obtain the soluble glucan oligomer of the present invention.

The inventive soluble glucan oligomer may be prepared in accordance with the following preferred embodiment.

For the present invention, above soluble glucan oligomer can be prepared by following procedure;
(a) 1^{st} step, the step culturing yeast IS2 (KCTC 0959BP) consisting that yeast IS2 (KCTC 0959BP) is cultured in liquid culture medium comprising 0.5-10 w/v% glucose, 0.1-5 w/v% yeast extract, 0.1-10 w/v% pepton;
(b) 2^{nd} step, the step obtaining yeast from yeast culture medium consisting that the yeast culture medium prepared from the first stage in a amount ranging from 0.1 to 10% (v/v) is inoculated to primary liquid culture medium comprising 0.5-10 w/v % glucose, 0.1-5 w/v % yeast extract, 0.01-2 w/v % ammonium sulfate, 0.001-1 w/v% potassium phosphate, and 0.001-1 w/v% magnesium sulfate in the pH ranging from 5.0 to 6.0, cultured for the period ranging from 12 hours to 48 hours at the speed ranging from 100 to 400 rpm, in the ventilating gas amount ranging from 0.3 to 3vvm, at the temperature ranging from 20 to 40° C. in growth media and then subjected to centrifugation to obtain yeast;
(c) 3^{rd} step, the step extracting wet beta-glucan from the cell wall of the yeast consisting that 1-10% sodium hydroxide solution is added to the yeast, dispersed, reacted for the period ranging from 30 minutes to 5 hours at the temperature ranging from 70 to 100° C., subjected to centrifugation to obtain dried cell mass (DCW) of yeast, of which process may be repeated at several times to pool, titrating the pH of the mass ranging from 4.0 to 5.0 using by strong acid such as hydrochloric acid and hydrogen sulfuric acid, dispersed again in sodium hydroxide solution, further reacting for 1 hour at 75° C., subjecting centrifugation to separate to sodium hydroxide solution and solid component; and finally washing and purifying the solid component to obtain wet beta-glucan;
(d) 4^{th} step, the step obtaining liquid phase of glucan oligomer consisting that distilled water at the amount equivalent to 1 to 10 times the volume of the glucan (v/v%) and beta-glucan hydrolyzing enzyme at the amount equivalent to 1/20 to ⅕ times of the glucan (v/w %) are added thereto, reacting for the period ranging from 6 to 24 hours at the temperature ranging from 30 to 80° C., recovering supernatant solution by centrifuging after quenching the reaction, filtering supernatant with ultra filtration membrane to obtain inventive soluble glucan oligomer solution having a molecular weight of less than 50,000;
(e) 5^{th} step, the step obtaining dried powder form of final soluble glucan oligomer consisting that the oligomer prepared from 4^{th} step is left alone for the period ranging from 12 hours to 48 hours at less than −70° C., and then subject to lyophilzation to obtain the powder form of soluble glucan oligomer of the present invention.

The soluble glucan oligomer prepared by above described procedure comprises glucan oligomer having a molecular weight of less than 50,000, preferably, ranging from 1,000 to 10,000.

It is the other object of the present invention to provide a pharmaceutical composition comprising soluble glucan oligomer derived from the cell wall of yeast variant strain (KTCT 0959BP) obtained by above described procedure as an active ingredient in an effective amount to treat and prevent mammal's diseases caused by the infection of influenza virus and transmissible gastroenteritis coronavirus, together with a pharmaceutically acceptable carrier thereof. It is the other object of the present invention to provide a process for preparing the soluble glucan oligomer as described above.

It is the other object of the present invention to provide the soluble glucan oligomer derived from yeast variant IS2 (KCTC 0959BP) prepared by the preparation as described above and it is the other object of the present invention to provide a pharmaceutical composition comprising soluble glucan oligomer derived from the cell wall of yeast variant strain (KTCT 0959BP) obtained by above described procedure as an active ingredient in an effective amount to treat and prevent mammal's diseases caused by the infection of influenza virus and transmissible gastroenteritis coronavirus disease, together with a pharmaceutically acceptable carrier thereof.

The composition of the present invention may be administered into human or domestic animal such as dog, cat, cattle and pig, and can be used in general administration method such as mixing with feed.

It is an object of the present invention to provide a use of a soluble glucan oligomer derived from yeast variant strain IS2 (KCTC 0959BP) for the preparation of therapeutic agent for treatment and prevention of the diseases caused by the infection of influenza virus and transmissible gastroenteritis coronavirus in human and mammal.

It is an object of the present invention to provide a method of treating or preventing influenza virus and transmissible gastroenteritis coronavirus disease in a mammal comprising the step of administering to said mammal an effective amount of composition comprising a soluble glucan oligomer derived from yeast variant strain IS2 (KCTC 0959BP), together with a pharmaceutically acceptable carrier thereof.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the composition of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing crude drug composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), suppository, or sterile injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active ingredients.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 0.1 to 1 g/kg by weight/day of the inventive composition of the present invention. The dose may be administered in a single or multiple doses per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

It is still another object of the present invention to provide a health care food comprising a composition essentially comprising a soluble glucan oligomer derived from yeast variant strain IS2 (KCTC 0959BP), together with a sitologically acceptable additive for preventing and improving mammal's diseases caused by the infection of influenza virus transmissible gastroenteritis coronavirus disease.

The health care food for preventing and alleviating mammal's diseases caused by the infection of influenza virus and transmissible gastroenteritis coronavirus disease could contain about 0.01 to 80 w/w%, preferably 1 to 50 w/w% of the above soluble glucan oligomer derived from yeast variant strain IS2 (KCTC 0959BP) of present invention based on the total weight of the composition.

The present invention provides a composition of the health care beverage for preventing and alleviating the diseases caused by the infection of influenza virus and transmissible gastroenteritis coronavirus disease in human and mammal comprising a soluble glucan oligomer derived from yeast variant strain IS2 (KCTC 0959BP).

Above inventive oligomer composition can be added to food and beverage for the preventing and alleviating the diseases caused by the infection of influenza virus and transmissible gastroenteritis coronavirus disease in human and mammal.

To develop for health care food, examples of addable food comprising above oligomer composition of the present invention are e.g., various food, beverage, bread, cookies, jam, candy, gum, tea, yogurt, vitamin complex, health improving food and the like, and can be used as power, granule, tablet, chewing tablet, capsule or beverage etc.

Inventive composition of the present invention has no toxicity and adverse effect, therefore, they can be used with safe.

Above described composition therein can be added to food, additive or beverage, wherein, the amount of above described oligomer in food or beverage may generally range from about 0.01 to 80 w/w % of total weight of food for the health care food composition and 0.02 to 30 g, preferably 0.3 to 5 g in the ratio of 100 ml of the health beverage composition.

Providing that the health beverage composition of present invention contains above described oligomer as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage. The inventive composition can be used as the mixing agent in the lactic acid bacteria-formulated beverage or paste and the like.

Above-mentioned component can be used independently or in combination.

The present invention provides a health care food comprising about 0.01 to 30 w/w % of the vitamin, oligosaccharides and dietary ingredients besides the composition of the present invention.

The ratio of the components is not so important but is generally range from about 0.01 to 30 w/w % per 100 w/w % present composition. Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, α-tocopherol, rosemary extract, vitamin C, licorice root extract, chitosan, tannic acid, phytic acid etc.

It will be apparent to those skilled in the art that various modifications and variations can be made in the compositions, use and preparations of the present invention without departing from the spirit or scope of the invention.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows the effect of soluble glucan oligomer on the NO (Nitric Oxide) production in alveolar macrophages.

Hereinafter, the present invention is more specifically explained by the following examples. However, it should be understood that the present invention is not limited to these examples in any manner.

### BEST MODE FOR CARRYING OUT THE INVENTION

The following Examples and Experimental Examples are intended to further illustrate the present invention without limiting its scope.

### EXAMPLE 1

### Culture of Yeast Variant IS2 and Harvest

Liquid medium containing 10 g/l of glucose, 6 g/l yeast extract, 3 g/l of ammonium sulfate ((NH₄)₂SO₄), 1.5 g/l of potassium phosphate (K₂PO₄) and 0.5 g/l of magnesium sulfate (MgSO₄.7H₂O) was used as a primary medium.

Liquid YPD medium (containing glucose 20 g/l, yeast extract 10 g/l and peptone 20 g/l) was used for inoculation and growth media containing 400g/l of glucose, 30 g/l yeast extract, 40 g/l of ammonium sulfate ((NH₄)₂SO₄), 15 g/l of potassium phosphate (K₂PO₄) and 5.7 g/l of magnesium sulfate (MgSO₄.7H₂O) was used for growth as a media.

After autoclaving the growth media, 100 ml of cultured yeast variant IS2(KCTC 0959BP) was seeded thereto, cultured in the rotating speed of 300 rpm and 1 vvm amount of ventilating gas, at 30° C. and pH 5.5 and finally 50-55 g/l of dried cell mass (DCW) of yeast was obtained through feed batch culture system.

### EXAMPLE 2

### Extraction of Beta Glucan from Yeast Variant IS2

80 g of DCW of yeast prepared in above Example 1, was suspended in 1,000 ml of 4% sodium hydroxide (NaOH) solution and then incubated at 95° C. for 1 hour. The incubated suspension was centrifuged at the speed of 2,000 rpm for 15 minutes to separate into NaOH solution part and solid part.

The separated solid part was suspended again in 2,000 ml of 3% sodium hydroxide solution, incubated at 75° C. for 3 hours and then centrifuged at the speed of 2,000 rpm for 15 minutes to separate into NaOH solution and solid part again.

The pooled solid part was adjusted to pH 4.5 with HCl, dispersed to the extent the final volume of 2,000 ml and incubated at 75° C. for 1 hour again. The incubated suspension was centrifuged at the speed of 2,000 rpm for 15 minutes to separate into NaOH solution part and solid part.

The solid part was washed 3 times with distilled water to obtain 160 g of wet beta glucan from the cell wall of yeast variant.

### EXAMPLE 3

### Preparation of Soluble Glucan Oligomer from O-glucan of Yeast Variant IS2

160 g of wet beta glucan prepared from Example 2 was put in 1,000 ml of flask and 480 ml of distilled water and beta O-glucanase at the amount equivalent to 1/10 of the glucan (v/w) were added thereto and incubated at 40° C. for 15 hours.

After stopping the reaction, the reaction mixture was centrifuged at 7,000 rpm for 15 minutes to collect the supernatant. The collected supernatant was filtered and the un-reacted enzymes were removed using by ultra filtration membrane (Filtron Co., MWCO 10K) to obtain the solution containing glucan oligomer having MW of less than 10,000 Dalton. After the solution had been left alone at −74° C. for overnight, the solution was lyophilized to produce 5.8 g of powder form of soluble glucan oligomer.

### EXPERIMENTAL EXAMPLE 1

### Effect of Soluble Glucan Oligomer on the NO (Nitric Oxide) Production in Alveolar Macrophage

To determine the effect of soluble glucan oligomer on the NO production in alveolar macrophage, following procedure was performed.

1-1. Cell culture

The alveolar macrophages was isolated from the lung of female pigs aged ranging from 1 to 3 weeks using by phosphate buffered saline (PBS, 2.56 g/l NaH₂PO₄.H₂O, 22.5 g/l Na₂HPO₄.7H₂O, 87.9 g/l NaCl, pH 7.2) and the isolated alveolar macrophages were suspended using by DMEM medium in 10% FBS and 2× antibacterial-antifungal solution in 75 cm² cell culture flask to be floated. The RNA was extracted from floated solution and PCR (polymerase chain reaction) was performed to confirm whether the macrophages is contaminated or not by seven types of porcine virus, i.e., porcine parvovirus, porcine circovirus type 2, porcine circovirus type 1, porcine reproductive and respiratory syndrome virus, Japanese encephalitis virus, encephalomyelocarditis virus and pseudorabies virus by the method disclosed in the literature (Jabrane et al., Can. Vet. J., 35, pp 86-92, 1994). After examining the contamination of alveolar macrophage for 12 hours, the plate was washed softly twice using by PBS solution before the inoculation of alveolar macrophage and the alveolar macrophage was detached from the surface of flask using by trypsin-EDTA solution. Finally, the detached alveolar macrophage was inoculated onto 24 well plates to be the concentration of 1×10⁶-10⁷/ml in each well plate.

1-2. Inducement of NO production

The soluble glucan oligomer prepared from Example 3 was dissolved in DMEM medium to be the concentration of 10 mg/ml, diluted in the manner of two fold dilution starting from 0.625 mg/ml to 5 mg/ml per well plate. After 36 hours, LPS (Lipopolysaccharide, Sigma, USA) solution diluted in the manner of two fold dilution starting from 100 ug/ml to 25 ug/ml per well plate was added thereto and incubated for 36 hours. After the incubation, the supernatants of the medium were collected to determine their NO (nitric oxide) production. Both of the group treated with only LPS and the group treat with nothing were used as a negative group. NO production was measured by conventional assay kit (nitrate/nitrite calorimetric assay kit, Cayman Chemical Co., USA).

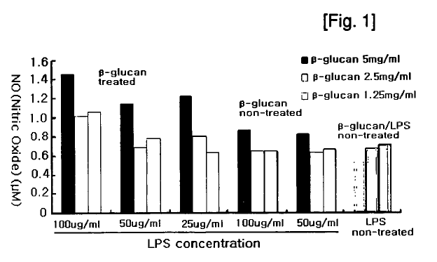

At the result, the experimental groups treated with soluble glucan oligomer derived from yeast variant strain IS2 showed more increase of NO production compared with that of negative control group and it was confirmed that the NO production by soluble glucan oligomer was increased with dose dependent manner (See FIG. 1).

### EXPERIMENTAL EXAMPLE 2

### Antiviral Activity of Soluble Glucan Oligomer on Swine Influenza Virus

To determine the antiviral activity of soluble glucan oligomer on swine influenza virus, both of the supernatant of the medium prepared in Experimental Example 1 treated with soluble glucan oligomer of the present invention and not treat with the soluble glucan oligomer was treated with swine viruses and inoculated into the alveolar macrophage cell. The antiviral effects of both were determined by observing the cytopathic effect (CPE) of viruses as follows.

2-1. Virus and Cell Preparation

Swine influenza virus was isolated from the lung of the pig infected with influenza virus and the TCID₅₀ (Tissue Culture Infective Dose 50) value of the pig was determined as 5×10^{7.5}/ml. NO for use in this experiment was prepared from the supernatant of alveolar macrophage medium obtained in Experimental Example 1-2. MDCK (Mardin-Darby Canine Kidney, ATCC, USA) cell, the derived from the dog kidney was used in the experiment.

2-2. Indirect Measurement of Antiviral Effect

The cells were prepared by incubating to proliferate into monolayer on 96 well plates. Both of 5 mg/ml of glucan treatment group and glucan/LPS co-treated group were used as NO treatment groups respectively and both of DMEM medium treatment group and glucan/LPS non-treatment group were used as negative control groups. Swine influenza virus was inoculated in various concentrations i.e., 10³, 10² and 10 TCID₅₀/well plate. After 24 and 36 hours of the inoculation, the cytopathic effects (CPE) of virus were determined using by inverted phase microscope. The mean value for serial three well was used for each group and the result was divided into 4 grades, i.e., strongest CPE (+++), moderate CPE (++), mild CPE (+) and no CPE (−) and compared by the method disclosed in the literature (Belaid et al., J. Med. Virol., 66 (2), pp 229-34, 2002).

At the result, glucan/LPS co-treatment groups showed highest antiviral effects among the experimental groups. Especially, it is confirmed that comparing with control group, glucan/LPS treated groups inhibit the CPE by virus by 70% and by about 30% after 36 hours. Moreover, the inhibiting effect maintains till 36 hours after the inoculation by 100% in the group treated with small amount of influenza virus in the concentration of 10² and 10¹ TCID₅₀ (See Table. 1).

TABLE 1

| | NO treatment group (n = 3) | | | | | | | | | Negative | | |
|---|---|---|---|---|---|---|---|---|---|---|---|---|
| | Glucan/LPS | | | Glucan treated | | | Positive control | | | control group | | |
| | treated group | | | group | | | group (n = 3) | | | (n = 3) | | |
| Hours | 10³* | 10² | 10¹ | 10³ | 10² | 10¹ | 10³ | 10² | 10¹ | 10³ | 10² | 10¹ |
| 24 | +** | − | − | ++ | + | − | +++ | +++ | +++ | − | − | − |
| 36 | ++ | − | − | ++ | + | − | +++ | +++ | +++ | − | − | − |

*TCID₅₀
**+++ (strong CPE), ++ (moderate CPE), + (mild CPE), − (no CPE)

2-3. Direct Measurement of Antiviral Effect

To determine the anti-viral activity of the soluble glucan oligomer, Each 5 mg/ml of the oligomer prepared in Example 3 was inoculated into 96 well plates together with swine viruses on MDCK cells of above Experimental Example 2-1 in the concentration of 10³, 10² and 10¹ TCID₅₀ per well plates. The group treated with only glucan was used as a Negative control groups. After 24 and 36 hours of the inoculation, the cytopathic effects (CPE) of virus were determined using by inverted phase microscope. The mean value for serial three well was used for each group and the result was divided into 4 grades, i.e., strongest CPE (+++), moderate CPE (++), mild CPE (+) and no CPE (−) and compared by the method disclosed in the literature (Belaid et al., J. Med. Virol., 66 (2), pp 229-34, 2002). Additionally, mixed solution of 5 mg/ml of the oligomer prepared in Example 3 and swine infuenza virus was inoculated onto the mono-layered cells to confirm the direct anti-viral effect of the glucan oligomer.

At the result, the group treated only glucan showed potent anti-viral effect by 100% till 24 hours after the inoculation compared with that of control group regardless the amount of virus. Both of the group treated with small amount of virus (10² and 10¹ TCID₅₀) and the group treated with high amount of virus (10³ TCID₅₀) showed 100% and 70% antiviral activity till 36 hours after the inoculation. (See Table. 2).

TABLE 2

| | Glucan/virus | | | | | | Negative | | | | | |
|---|---|---|---|---|---|---|---|---|---|---|---|---|
| | treated group | | | Positive control | | | control group | | | Glucan treated | | |
| | (n = 3) | | | group (n = 3) | | | (n = 3) | | | group (n = 3) | | |
| Hours | 10³* | 10² | 10¹ | 10³ | 10² | 10¹ | 10³ | 10² | 10¹ | 10³ | 10² | 10¹ |
| 24 | −** | − | − | +++ | +++ | +++ | − | − | − | − | − | − |
| 36 | + | − | − | +++ | +++ | +++ | − | − | − | − | − | − |

*TCID₅₀
**+++ (strong CPE), ++ (moderate CPE), + (mild CPE), − (no CPE)

### EXPERIMENTAL EXAMPLE 3

### Antiviral Effect of Soluble Glucan Oligomer on TGEV (Transmissible Gastroenteritis Coronavirus)

To determine the anti-viral effect of soluble glucan oligomer on TGEV (transmissible gastroenteritis coronavirus), following procedure was performed.

3-1. Virus and Cell Preparation

Miller strain of Transmissible gastroenteritis coronavirus (TGEV), a coronavirus was procured by Seoul National University disclosed in the literature (Kim B. and Chae C., J. Comp. Path., 126, pp 30-37, 2002) and the TCID₅₀ value of TGEV before the experiment was determined to be 1×10⁶/ml.

Pig testicular cell (ATCC, USA) was isolated from the testis of pig to use in this experiment.

3-2. Indirect Measurement of Antiviral Effect

To determine the indirect antiviral effect of soluble glucan oligomer of the present invention, the culture supernatant of alveolar macrophage prepared in Example 1-2 was mixed with coronavirus and the mixture was inoculated into the monolayer on 96 well plates. Further procedures were performed in similar to the methods disclosed in Example 2-2.

At the result, the culture supernatant prepared from alveolar macrophage treated with both of glucan and LPS showed highest antiviral activity among the groups. The CPE value of the group treated with both of glucan and LPS in high amount of virus (103 TCID₅₀) showed potent anti-viral effect by about 70% at 24 hours after the inoculation compared with that of control group and by about 30% at 36 hours after the inoculation compared with that of control group (See Table. 3).

TABLE 3

| | NO treatment group (n = 3) | | | | | | | | | Negative | | |
|---|---|---|---|---|---|---|---|---|---|---|---|---|
| | Glucan/LPS | | | Glucan treated | | | Positive control | | | control group | | |
| | treated group | | | group | | | group (n = 3) | | | (n = 3) | | |
| Hours | 10³* | 10² | 10¹ | 10³ | 10² | 10¹ | 10³ | 10² | 10¹ | 10³ | 10² | 10¹ |
| 24 | +** | + | + | ++ | + | + | +++ | +++ | +++ | − | − | − |
| 36 | ++ | ++ | ++ | ++ | ++ | ++ | +++ | +++ | +++ | − | − | − |

*TCID₅₀
**+++ (strong CPE), ++ (moderate CPE), + (mild CPE), − (no CPE)

3-3. Direct Measurement of Antiviral Effect

To determine the direct antiviral effect of soluble glucan oligomer of the present invention, Each 5 mg/ml of the oligomer prepared in Example 3 was mixed with swine viruses and the mixture was inoculated into 96 well plates. Further procedures were performed in similar to the methods disclosed in Example 2-3.

At the result, the group treated with only soluble glucan oligomer showed highest antiviral activity among the groups. The CPE value of the group treated with only soluble glucan oligomer showed potent anti-viral effect by about 70% in highest amount of virus (103 TCID₅₀) at 24 hours, about 30% in high amount of virus (10³, 10³TCID₅₀) and about 70% in small amount of virus (10¹ TCID₅₀) at 36 hours the inoculation compared with that of control group (See Table. 4).

TABLE 4

| | Glucan/virus | | | | | | Negative | | | | | |
|---|---|---|---|---|---|---|---|---|---|---|---|---|
| | treated group | | | Positive control | | | control group | | | Glucan treated | | |
| | (n = 3) | | | group (n = 3) | | | (n = 3) | | | group (n = 3) | | |
| Hours | 10³* | 10² | 10¹ | 10³ | 10² | 10¹ | 10³ | 10² | 10¹ | 10³ | 10² | 10¹ |
| 24 | +** | + | + | +++ | +++ | +++ | − | − | − | − | − | − |
| 36 | ++ | ++ | + | +++ | +++ | +++ | − | − | − | − | − | − |

*TCID₅₀
**+++ (strong CPE), ++ (moderate CPE), + (mild CPE), − (no CPE)

### EXPERIMENTAL EXAMPLE 4

### Toxicity Test

4-1. Methods

The acute toxicity tests on ICR mice (mean body weight 25±5 g) and Sprague-Dawley rats (235±10 g, Jung-Ang Lab Animal Inc.) were performed using the oligomer of the Example 3. Four group consisting of 10 mice or rats was administrated orally with 4 mg/kg, 40 mg/kg, 400 mg/kg and 4,000 mg/kg of test sample or solvents (0.2 ml, i.p.) respectively and observed for 2 weeks.

4-2. Results

There were no treatment-related effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of Powder

| | | |
|---|---|---|
| Dried powder of Example 3 | 50 | mg |
| Lactose | 100 | mg |
| Talc | 10 | mg |

Powder preparation was prepared by mixing above components and filling sealed package.

Preparation of Tablet

| | | |
|---|---|---|
| Dried powder of Example 3 | 50 | mg |
| Corn Starch | 100 | mg |
| Lactose | 100 | mg |
| Magnesium Stearate | 2 | mg |

Tablet preparation was prepared by mixing above components and entabletting.

Preparation of Capsule

| | | |
|---|---|---|
| Dried powder of Example 3 | 50 | mg |
| Corn Starch | 100 | mg |
| Lactose | 100 | mg |
| Magnesium Stearate | 2 | mg |

Tablet preparation was prepared by mixing above components and filling gelatin capsule by conventional gelatin preparation method.

Preparation of Injection

| | |
|---|---|
| Dried powder of Example 3 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of Liquid

| Dried powder of Example 3 | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of Health Care Food

| Dried powder of Example 3 | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B6 | 0.5 | mg |
| Vitamin B12 | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in many ways. Such variations are not to be regarded as a departure from the spirit and scope of the present invention.

Preparation of Health Beverage

| | | |
|---|---|---|
| Dried powder of Example 3 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1 000in ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described above, the soluble glucan oligomer having a M.W. ranging from 1,000 to 10,000 prepared by treating insoluble beta-glucan isolated from the cell wall of yeast variant IS2 with commercially available beta-glucan hydrolyzing enzymes, showed potent inhibiting activity of influenza virus and transmissible gastroenteritis coronavirus, therefore, it can be used as the therapeutics or health care food for treating and preventing the diseases infected by influenza virus and transmissible gastroenteritis coronavirus.

What is claimed is;:

1. A use of a soluble glucan oligomer derived from yeast variant strain IS2 (KCTC 0959BP) for the preparation of therapeutic agent for treatment and prevention of the diseases caused by the infection of influenza virus and transmissible gastroenteritis coronavirus in human and mammal.

2. The use according to claim 1 wherein said influenza virus is influenza virus A, B, C₄ or swine influenza virus.

3. The use according to claim 1 wherein said diseases infected by influenza virus is influenza, common cold, laryngopharyngitis, bronchitis or pneumonia.

4. The use according to claim 1 wherein said disease infected by transmissible gastroenteritis coronavirus is common cold, severe acute respiratory disease, porcine epidemic diarrhea (PED) or transmissible gastro enteritis (TGE).

5. The use according to claim 1 wherein said mammal is human and domestic animals.

6. A pharmaceutical composition comprising soluble glucan oligomer derived from the cell wall of yeast variant strain (KTCT 0959BP) obtained by treating insoluble beta-glucan with enzyme, for treating and preventing the diseases caused by the infection of influenza virus and transmissible gastroenteritis coronavirus in human and mammal.

7. A health care food comprising the soluble glucan oligomer derived from yeast variant strain IS2 (KCTC 0959BP) as an active ingredient, together with a sitologically acceptable additive for the prevention and improvement of mammal's diseases caused by the infection of influenza virus and transmissible gastroenteritis coronavirus.
